(19) United States
(12) Patent Application Publication
(10) Pub. No.: US 2004/0143205 A1
(43) Pub. Date: Jul. 22, 2004

(54) Wrist brace with metacarpal phalangeal block

(75) Inventor: Dana T. Ressel, Upland, CA (US)

Correspondence Address: Thomas R. Juettner, Greer, Burns & Crain, Ltd., Suite 2500, 300 South Wacker Drive, Chicago, IL, 60606, US

(73) Assignee: d j Orthopedics, LLC

(21) Appl. No.: 10/348,857

(22) Filed: Jan. 21, 2003

(51) Int. Cl. A61F 5/00

(52) U.S. Cl. 602/21; 602/20

(57) ABSTRACT

A wrist brace with a metacarpal phalangeal block employes a soft goods body with appropriate stays for supporting a hand and a wrist without enclosing the fingers or thumb, and a stay affixed to the body and including a first portion extending to the metacarpal phalangeal joint and a blocking portion overlying the proximal phalange of at least one finger. The blocking portion of the stay prohibits extension of the proximal phalange of the finger beyond a blocked position substantially normal to the palm of the hand while at the same time permitting flexion and extension of the finger between the blocked position and the palm of the hand. The wrist brace is particularly useful in the treatment of fractures of the metacarpal bones and other bones of the hand and fingers.

### FIELD OF THE INVENTION

This invention relates to removable hand and wrist braces or splints used when one or more of the metacarpal bones or other bones of the hand and/or fingers is or are broken.

### BACKGROUND OF THE INVENTION

When the long bone of the hand which leads to the little finger is broken, often referred to as a “boxer's fracture”, a plaster east is normally used to tightly encompass a substantial portion of the wrist, hand and fingers. This cast usually encompasses a substantial portion of the little finger (fifth finger), adjacent finger (fourth finger), palm of the hand and wrist. Therefore, the cast allows little, if any, beneficial use of the hand or fingers. In addition, this cast cannot be removed until total mending of the break. Due to the lack of mobility during this period, the wrist, hand and fingers become atrophied and temporarily dysfunctional. If atrophy occurs, the patient must often undergo extensive rehabilitative treatment under the supervision of a physician or a physical therapist in order to exercise and strengthen the atrophied limb. Rehabilitation therapy, although necessary, is a costly and time consuming, and frequently painful procedure.

Many devices and orthotics relating to hand and finger injuries have been proposed as an alternative to plaster casts in an effort to cut down on the weight, decrease the bulkiness or improve the comfort for the user, and permit some use of the hand.

U.S. Pat. No. 5,058,576, for example, discloses an adjustable and removable splint for immobilizing the wrist, the outer (ulnar) side of the hand and the fourth and fifth fingers while permitting beneficial use of the remainder of the hand, fingers and thumb. The splint includes a hand and wrist encompassing sleeve, a plurality of stays secured to the sleeve for engagement with the palmar, dorsal and ulnar surfaces of the hand and both the palmar and dorsal surfaces of the fourth and fifth fingers over their full lengths, and straps or like means for securing the splint to the wrist, hand and fourth and fifth fingers to immobilize the wrist, the outer portion of the hand and the fourth and fifth fingers. The brace, like other prior art braces and plaster casts, immobilizes the fourth and fifth fingers, which may result in atrophy and dysfunction of the fingers and the muscles and tendons controlling the same, thus requiring post-healing physical therapy and rehabilitation.

### SUMMARY OF THE INVENTION

The present invention is predicated on the ascertainment and practical reality that for healing boxers' fractures and similar injuries of the hand and fingers, the important factors are (1) to prevent the fourth and fifth fingers from extension beyond a position normal to, i.e., at a right or substantially ninety degree angle to, the palm of the hand, and (2) to premit limited movement of the fourth and fifth fingers to maintain the physical tone and well-being of the fingers and the related muscles and tendons, to minimize or prevent atrophy, and to minimize post-healing therapy and rehabilitation.

In accordance with the invention, proper healing and mending of boxer and like fractures is promoted by blocking the proximal phalanges of the fourth and fifth fingers from extension beyond a position normal to the palm of the hand while permitting flexion or bending of the fingers toward and away from the palm of the hand. In this manner, limited movement of the metacarpal joint and the tendons, muscles and joints of the fourth and fifth fingers is accommodated to inhibit atrophy and dysfunction of the muscles and tendons. Consequently, the need for rehabilitative physical therapy following healing is greatly reduced if not eliminated altogether.

Pursuant to the invention, an adjustable and removable wrist brace is provided with one or more stays for maintaining the wrist and hand in proper positional relationship for optimum healing of a fracture of the hand and with an additional stay extending from the hand portion of the brace to a position overlying the proximal phalanges of the fourth and fifth fingers for blocking (preventing) extension of the proximal phalanges and thus the entirety of the fourth and fifth fingers, beyond a position substantially normal to the palm of the hand.

In light of the described function, the additional stay provided pursuant to the invention is herein defined as a metacarpal phalangeal block or MP block.

An adjustable strap is preferably associated with the MP block for alternative adjustment between a first position wherein the entirety of the fourth and fifth fingers are free to be flexed from the blocked position toward and away from the palm and a second position wherein the proximal phalanges are secured to the block and only the middle and distal phalanges are free for flexion and extension.

In a first embodiment of the invention, the stay comprising the MP block is secured to the ulnar side of the wrist brace and extends along the ulnar or outer side of the hand and proximal phalange of the fifth or little finger, and then over the proximal phalanges of the fifth and fourth fingers, or alternatively, over the proximal phalanges of the fifth, fourth, third and second fingers.

In a second embodiment of the invention, the stay comprising the MP block is secured to the palm of the wrist brace and extends between the proximal phalanges of the third and fourth fingers and then over the proximal phalanges of the fourth and fifth fingers. Alternatively, in the second embodiment, the stay maybe reversed to extend over the proximal phalanges of the second and first fingers. As a further alternative, the stay maybe formed of “T” shape with one branch of the “T” extending over the proximal phalanges of the fourth and fifth fingers and the other branch of the “T” extending over the proximal phalanges of the third and second fingers.

In all of its embodiments, the MP block of the invention provides the advantages of preventing excessive and potentially harmful extension of the proximal phalanges of the fingers, thereby to promote proper healing, while at the same time permitting limited flexion and extension of the fingers between the MP block and the palm of the hand to maintain the well-being and physical tone of the associated muscles and tendons to minimize atrophy and the need for post-healing rehabilitative therapy.

These and other objects and advantages of the invention will be become apparent to a person of reasonable skill in the art from the following detailed description, as considered in conjunction with the accompanying drawings.

### BRIEF DESCRIPTION OF THE DRAWINGS

FIG. 1 is a view in elevation of the palmar side of one embodiment of the wrist brace of the invention;

FIG. 2 is a view in elevation of the radial or thumb side of the wrist brace illustrated in FIG. 1;

FIG. 3 is a view in elevation of the dorsal and ulnar surfaces of the wrist brace illustrated in FIG. 1;

FIG. 4 is a fragmentary view of the wrist brace illustrated in FIG. 1 showing in cross section the MP block of the invention and the mode of mounting the MP block to the wrist brace pursuant to the invention;

FIG. 5 is a view in elevation of a soft goods member used in the FIG. 1 embodiment of the invention for securing the stay of the MP block to the brace and also providing an adjustable strap for controlling the degree of movement permitted of the phalanges of the confined fingers;

FIG. 6 is a fragmentary view in elevation of the wrist brace illustrated in FIG. 1 and showing an alternative position of adjustment of the adjustable strap for restricting the degree of movement of the proximal phalanges of the confined fingers; and

FIG. 7 is a view in elevation of the palmar side of a second embodiment of the wrist brace of the invention.

### DETAILED DESCRIPTION OF PREFERRED EMBODIMENTS

The following is a detailed description of certain embodiments of the invention presently deemed by the inventor to be the best mode of carrying out his invention.

The wrist and hand supporting or immobilizing parts of the wrist brace employed in practice of the invention maybe of any conventional or known construction suitable for the use herein described and capable of modification to incorporate therein the metacarpal phalangeal block (MP block) of the invention.

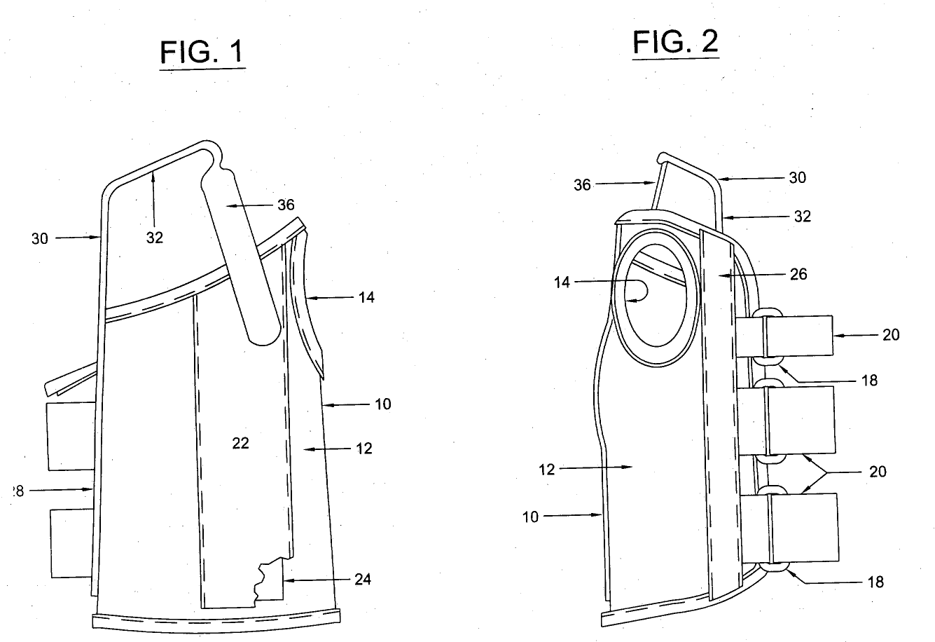

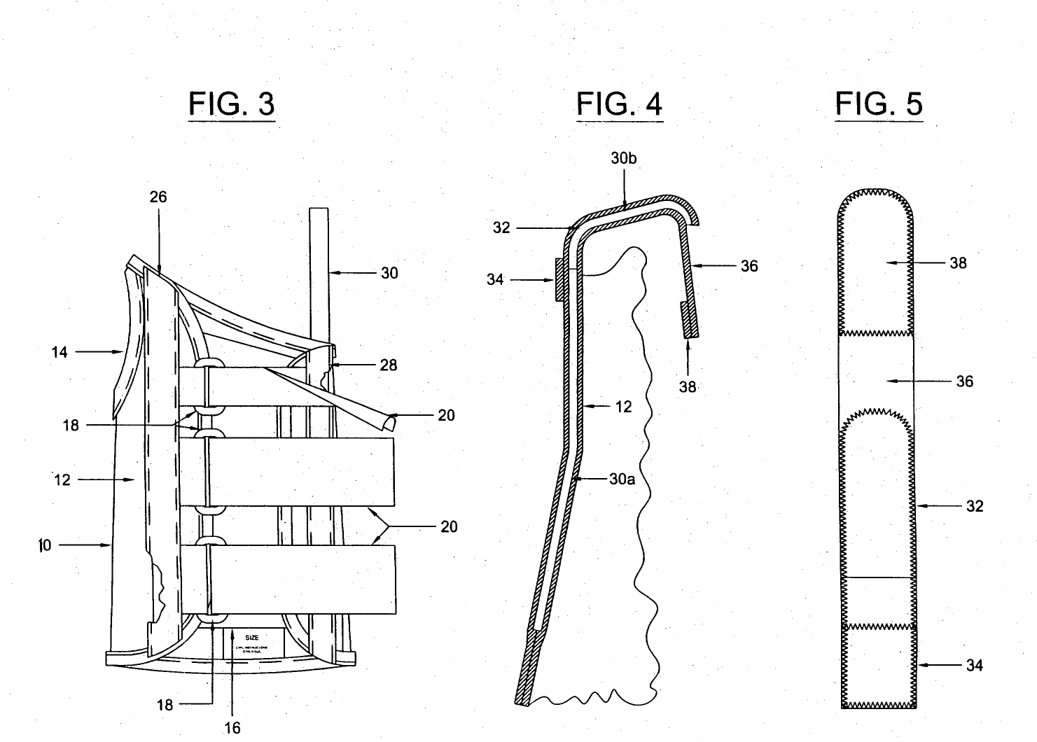

Referring to FIGS. 1-3, a known wrist brace 10 suitable for the purpose is comprised of a soft goods body 12 formed, for example, from a laminate of lycra and foam and shaped to cover the palm and the ulnar and radial surfaces of the hand, wrist and forearm. A thumb hole 14 is provided in the body 12 to accommodate free and complete movement of the thumb. A sheet 16 (FIG. 3) of stockingette material bridges between the sides edges of the body 12 for extension over the dorsal surface of the hand and wrist to provide a comfortable fit of the body on the hand and wrist. Buckles 18 are provided on one edge of the body 12 for reception of straps 20 attached to the other edge of the body for extension over the dorsal surface of the hand and wrist for firmly securing the brace to the hand and wrist.

The body 12 is provided with a number of pockets for reception of plastic or metal stays that serve to rigidify the brace and establish the proper positional relationship between the hand, wrist and forearm to facilitate healing of a fracture or other injury to the wrist, hand and/or fingers. The brace illustrated in the drawings includes a palmar pocket 22 for reception of a palmar stay 24 that extends substantially from the metacarpal phalangeal joints of the hand over the palmar surface of the hand,, wrist and forearm to substantially immobilize the wrist; a radial pocket 26 for reception of a radial stay (not shown) of substantially the same length as the stay 24 that extends along the radial or thumb side of the hand, wrist and forearm: and an ulnar stay pocket 28 that extends along the outer or ulnar surface of the hand, wrist and forearm.

The stay pocket 28 would normally receive a stay of substantially the same length as the stay 24, i.e., of a length to extend along the outer surface of the hand from the MP joint to the forearm. However, in the embodiment of the invention illustrated in FIGS. 1-5, the pocket 28 receives an elongated stay 30 that serves the usual function of a ulnar stay and in addition provides a MP block for preventing extension of the proximal phalanges of the fourth and fifth fingers, and if desired, the second, or the first and second fingers, beyond a position substantially normal to the palm of the hand. The stay is preferably a strip of aluminum about ½ inch wide.

Referring to FIGS. 1 and 4, the stay pocket 28 is defined between the outer surface of the body 12 of the brace and a strip of material, preferably of the hook and loop fastener type, which is secured to the body at its sides and forearm end, leaving the MW end open for insertion and removal of a body portion 30a of the stay 30. The body portion 30a is appropriately configured or curved to conform to the shape of the outer surface of the hand, wrist and forearm. The stay extends upwardly from the pocket 28 and is curved inwardly in outwardly spaced relation to the MP end of body 12 to form a finger blocking portion 30b that overlies and defines a space to be occupied by the proximal phalanges of the fourth and fifth fingers when the brace is fitted to a hand.

As an alternative, the blocking portion 30b of the stay maybe extended to overlie the proximal phalange of the third finger of the hand or to overlie the proximal phalanges of the second and third fingers of the hand, as well as the proximal phalanges of the fourth and fifth fingers of the hand.

The blocking portion 30b of the stay 30 is preferably covered with a soft goods sleeve 32 that is fitted at its proximal or MP end with a tab 34 of hook and loop fastener material matable with and fastenable to the strip of material forming pocket 28. The tab 34 locks the stay 30 in the pocket 28 and thus to the body 12. Also, the arrangement of the tab 34 relative to pocket 28 accommodates a degree of adjustment of the stay relative to the MP end of the body 12 thereby to vary the size of the space between the block and the body to adapt the block for fingers of different sizes and/or to accommodate variation in the degree of movement permitted of the proximal phalanges of the fourth and fifth fingers.

A flexible strap 36 extends from the distal end of sleeve 32 for passage between the third and fourth fingers of the hand and for attachment to the body 12 of the brace. The strap engages the lateral side of the fourth finger to retain the proximal phalanges of the fourth and fifth fingers aligned with and in proper positional relationship to the blocking portion 30b of the stay 30. The strap also inhibits or prohibits abduction and adduction of the fourth and fifth fingers. A tab 38 of hook and loop fastener material is fixed to the end of the strap 36 for attaching the strap to matable hook and loop material on the brace body 12.

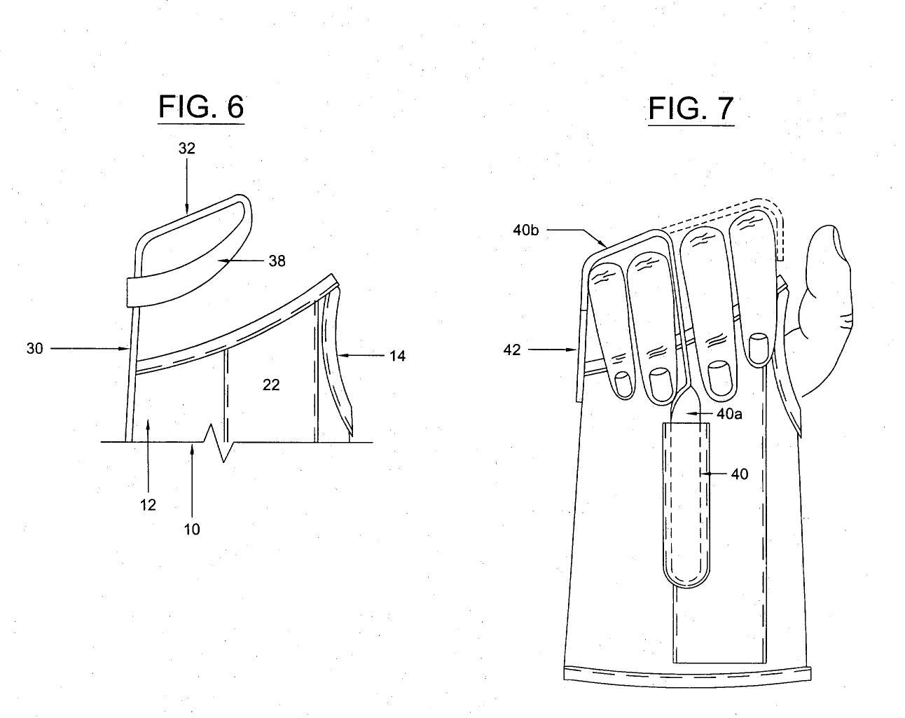

The strap 36 is adapted to extend between the third and fourth fingers and to be affixed to the material defining the palmar pocket 22 as shown in FIG. 1. In this position of the strap, all three phalanges of the fourth and fifth fingers can be flexed and extended back and forth between the block 30b and the palm of the hand. Alternatively, the strap can be wrapped around the palmar surfaces of the proximal phalanges of the fourth and fifth fingers and secured by the tab 38 to the material forming pocket 28, as shown in FIG. 6. In this position, the proximal phalanges of the fourth and fifth fingers are held to the block 30b and only the middle and distal phalanges are moveable between the blocked position and the palm of the hand. Thus, different modes of treatment of an injured hand are accommodated.

Referring to FIG. 7, a second embodiment of the invention utilizes the same wrist brace as described in conjunction with FIGS. 1-6 with the exception that the MP block is affixed to the palmar surface of the brace. A stay 40 for forming an MP block has a width of about ½ inch and is comprised of a body portion 40a and a blocking portion 40b that is twisted at right angles to the body portion 40a. The blocking portion 40b is adapted to extend between the third and fourth fingers of the hand and is then bent at the MP joint to about a right angle to overlie the proximal phalanges of two fingers. With this embodiment of the invention, the MP block may overlie the proximal phalanges of the fourth and fifth fingers as shown in solid lines in FIG. 7, or maybe reversed to overlie the proximal phalanges of the second and third fingers as shown in dotted lines in FIG. 7. Alternatively, the blocking portion may be T shaped to overlie the proximal phalanges of all four fingers.

The body portion 40a of the stay 40 maybe inserted in the palmar pocket 22 of the brace, and if desired may comprise the palmar stay. Alternatively, the body portion 40a may be housed in a soft goods sleeve adapted to be detachably affixed by hook and loop fasteners to the strip of material defining the pocket 22.

The blocking portion 40b of the stay 40 is preferably housed in a soft good sleeve like the sleeve 32 with a hook and loop fastener tab like the tab 34 for detachable securement to the material housing the body portion 40a. Such sleeve also includes a strap 42 for detachable securement to the ulnar surface of the body of the brace, as shown in FIG. 7, or for wrapping around the palmar surface of the proximal phalanges and attachment to the sleeve housing the part of the stay 40 that passes between the third and fourth fingers for holding the proximal phalanges of the fourth and fifth fingers to the block 40b.

The FIG. 7 embodiment of the invention thus provides essentially the same advantages as the embodiment of FIGS. 1-6.

Both embodiments of the invention provide a wrist brace including a MP block prohibiting extension of the proximal phalanges of two or more fingers, and thus the entirety of the fingers, beyond a position normal to the palm of the hand, while at the same time permitting flexion and extension of the fingers back and forth between the block and the palm of the hand. The blocked fingers may thus be exercised to maintain the well-being and physical tone of the related muscles and tendons and to minimize atrophy during healing of an injured hand, and to minimize post-healing therapy and rehabilitation.

The objects and advantages of the invention have thus been shown to be attained in a convenient, practical and facile manner.

While certain presently preferred embodiments of the inventions have been herein illustrated and described, it is to be appreciated that various changes, rearrangements and modifications may be made therein without departing from the scope of the invention as defined by the appended claims.

What is claimed is:

1. A hand and wrist brace for supporting a hand and wrist and for limiting movement of the proximal phalange of one or more fingers comprising
a soft goods body for at least partially enclosing the wrist and hand, and
a stay affixed to said body and including a portion extending to the metacarpal phalangeal joint and overlying the proximal phalange of at least one finger,
said portion of said stay prohibiting extension of the proximal phalange beyond a position substantially normal to the palm of the hand while permitting flexion and extension of the finger between said position and the palm of the hand.

2. A brace as set forth in claim 1 including a flexible strap associated with said portion of said stay and attachable to said body for retaining the proximal phalange of the finger aligned with said portion.

3. A brace as set forth in claim 2 wherein said strap is engageable with a lateral side and the palmar surface of the proximal phalange of the finger for holding the proximal phalange of the finger to said portion of said stay while permitting flexion and extension of the middle and distal phalanges of the finger between said position and the palm of the hand.

4. A brace as set for in claim 2 wherein said strap engages a lateral side of a proximal phalange of the finger for aligning the proximal phalange with said portion of said stay while permitting flexion and extension of the three phalanges of the finger between said position and the palm of the hand.

5. A brace as set forth in claim 1 including a soft goods sleeve encompassing said portion of said stay and including means for detachably and adjustably securing the same to said body for accommodating adjustment of the position of said portion relative to said body.

6. A brace as set forth in claim 1 wherein said stay extends along the ulnar surface of said body and said portion of said stay overlies the proximal phalange of at least the fifth finger.

7. A brace as set forth in claim 6 wherein said portion of said stay overlies the proximal phalanges of the fourth and fifth fingers.

8. A brace as set forth in claim 6 wherein said portion of said stay overlies the proximal phalanges of the third, fourth and fifth fingers.

9. A brace as set forth in claim 6 wherein said portion of said stay overlies the proximal phalanges of the second, third, fourth and fifth fingers.

10. A brace as set forth in claim 1 wherein said stay is affixed to the palmar surface of said body and is extendible between the third and fourth fingers and said portion of said stay overlies the proximal phalange of at least one of said fingers.

11. A brace as set forth in claim 10 wherein said portion of said stay is extendible over the proximal phalanges of the fourth and fifth fingers, or the first and second fingers, or all four of said fingers.

12. A hand and wrist brace for supporting a hand and wrist and for limiting movement of the proximal phalange of one or more fingers comprising
a soft goods body for at least partially enclosing the wrist and hand but not enclosing the fingers or the thumb, said body including a stay receiving pocket extending along the ulnar surface of the body, and
a stay having a mounting portion within said pocket and extending to the metacarpal phalangeal joint of the hand and a blocking portion extending inwardly over the proximal phalanges of at least the fourth and fifth fingers,
said blocking portion prohibiting extension of the proximal phalanges of the fourth and fifth fingers beyond a blocked position substantially normal to the palm of the hand while permitting flexion and extension of the fingers between the palm of the hand and said blocked position.

13. A brace as set forth in claim 12 including a flexible strap associated with said blocking portion of said stay, said strap being extendable between the third and fourth fingers and attachable to said body for engaging the side of the proximal phalange of the fourth finger and retaining the proximal phalanges of the fourth and fifth fingers aligned with said blocking portion.

14. A brace as set forth in claim 13 wherein said strap is attachable to the palmar surface of said body for permitting flexion and extension of all three phalanges of the fingers between the palm of the hand and said blocked position.

15. A brace as set forth in claim 13 wherein said strap is engagable with the side of the fourth finger and the palmar surfaces of the proximal phalanges of the fourth and fifth fingers for holding the proximal phalanges of the fourth and fifth fingers to said blocking portion while permitting flexion and extension of the middle and distal phalanges of the fingers between said blocked position and the palm of the hand.

16. A brace as set for in claim 12 including a soft goods sleeve encompassing said blocking portion of said stay and including means for detachably and adjustably securing the same to said pocket for securing said stay in said pocket and for accommodating adjustment of the position of said blocking portion relative to said body.

17. A hand and wrist brace for supporting a hand and wrist and for limiting movement of the proximal phalange of one or more fingers comprising
a soft goods body for at least partially enclosing the wrist and hand but not enclosing the fingers or the thumb, said body including a stay receiving pocket on the palmar surface of the body, and
a stay having a mounting portion within said pocket and extending between the proximal phalanges of the third and fourth fingers and a blocking portion extending over the proximal phalanges of at least two adjacent fingers,
said blocking portion prohibiting extension of the proximal phalanges beyond a blocked position substantially normal to the palm of the hand while permitting flexion and extension of the two fingers between the palm of the hand and said blocked position.

18. A brace as set forth in claim 17 wherein said blocking portion of said stay extends substantially normal to the mounting portion and is of a length to overlie the proximal phalanges of two fingers and said mounting portion is reversably mountable in said pocket for positioning said blocking portion over the proximal phalanges of either the fourth and fifth fingers or the first and second fingers.
